(19) United States
(12) Patent Application Publication
(10) Pub. No.: US 2008/0070980 A1
(43) Pub. Date: Mar. 20, 2008

(54) Use Of Beta-Cryptoxanthin

(76) Inventors: Anne Eichinger, Mulhouse (FR); Regina Goralczyk, Grenzach-Wyhlen (DE); Karin Wertz, Rheinfelden (DE); Adrian Wyss, Basel (CH)

Correspondence Address: NIXON & VANDERHYE, PC, 901 NORTH GLEBE ROAD, 11TH FLOOR, ARLINGTON, VA, 22203, US

(21) Appl. No.: 11/596,618

(22) PCT Filed: May 10, 2005
(86) PCT No.: PCT/EP05/05030

§ 371 Date: Nov. 15, 2006

(30) Foreign Application Priority Data
May 18, 2004 (EP) 04.011743.4

(51) Int. Cl. A61K 31/045; A23K 1/16; A61K 31/355; A61P 43/00; A23L 1/303

(52) U.S. Cl. 514/458; 426/2; 426/73; 514/729

(57) ABSTRACT

β-Cryptoxanthin and esters thereof may find use for promoting an increased protein formation and/or prevention of loss of proteins in humans and animals.

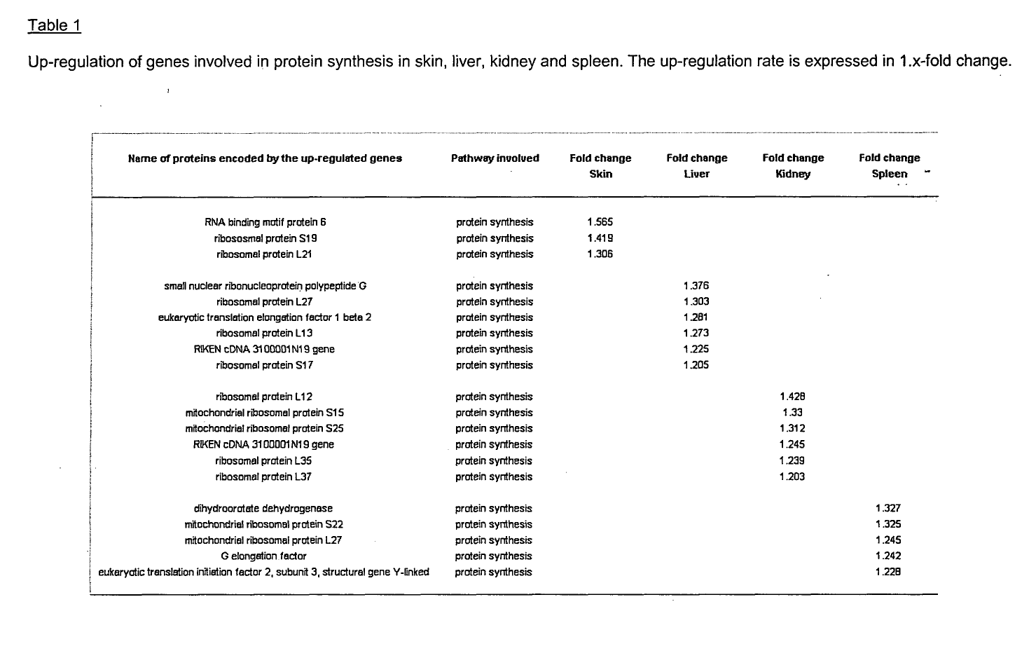

The present invention relates to the use of β-cryptoxanthin in human and in animals. More particularly the present invention relates to use of β-cryptoxanthin in the manufacture of a composition for promoting an increased protein formation and/or prevention of loss of proteins. In a further aspect, the present invention relates to a method for promoting an increased protein formation and/or prevention of loss of protein in human or an animal which comprises administering to said human or animal an effective amount of β-cryptoxanthin.

The term β-cryptoxanthin as used herein comprises β-cryptoxanthin either from natural source or synthetically prepared β-cryptoxanthin. β-Cryptoxanthin (more specifically, (all-E) β-cryptoxanthin) from natural source may contain β-cryptoxanthin esters with saturated and unsaturated fatty acids, (mainly laurate, myristate, palmitate, stearate, linolate,) as well as the isomers (preferably 7′,9′,11′ and 13′β-cryptoxanthin) which are included also for use in the present invention. In a preferred aspect, synthetically prepared (all-E)-β-cryptoxanthin is used for the purposes of the invention.

The present invention especially relates to the use of β-cryptoxanthin in the manufacture of a composition for enhancement and increase of protein synthesis in tissue (i.e. liver, skin, kidney, muscle etc.) of humans and animals in all conditions where an increased protein formation in the body is needed or desirable, e.g., in adverse health conditions, such as tumor cachexia, eating disorders (e.g., bulimia and anorexia), chronic diseases, such as chronic heart failure, chronic obstructive pulmonary disease, chronic bowel diseases (e.g. Crohn's disease), chronic degenerative diseases e.g., osteoporosis, rheumatoid arthritis, osteoarthritis; as well as to generally foster growth and development, in sports and workout, convalescence, and during aging for prevention and treatment of sarcopenia. In addition, the present invention relates to the use of β-cryptoxanthin in the manufacture of a composition enhancing performance and promoting growth of farm animals, pets and competitive sport animals. The invention also comprises the use of β-cryptoxanthin as an agent in the treatment of conditions where an increased protein formation in the body is needed or desirable.

In particular, β-cryptoxanthin can find use in accordance with the present invention to enhance and/or to potentiate the protein synthesis in case of syndromes and pathologies inducing protein wasting, or in the prophylactic supplementation of healthy subjects. Further examples of adverse health conditions where β-cryptoxanthin may find use in accordance with the invention are states of infections with the Human Immunodeficiency Virus when patients show the first signs of the Acquired ImmunoDeficiency Syndrome (AIDS), cases of recent transplants, and chronic alcohol abuse. β-Cryptoxanthin may also be used in bedridden people for prevention and treatment of immobilization induced muscle loss, or in people suffering from other long lasting infectious diseases, like hepatitis, Epstein-Barr Virus and Varicella zoster infections. Further, β-cryptoxanthin may be used in accordance with the invention in the improvement of the general health status of convalescent patients, e.g., after second or third degree burns, myocardial infarction, and organ transplantation. It has been found also that the promotion of protein formation by β-cryptoxanthin in accordance with the present invention is even more effective when bile salts (e.g., sodium cholate, glycocholate, taurocholate, glycodeoxycholate) are present in the gut. Thus, in another aspect of the invention, β-cryptoxanthin is used in combination with choleretic stimulants, or by addition of bile salts to the diet. Examples of choleretic stimulants for use in the present invention are coffee, green or black tea, herbs such as camomile, rosemary, mint, mate, milk thistle, lavender, fennel, artichoke as teas or extracts thereof, or polyunsaturated fatty acids, suitably as capsules. In still another aspect of the invention, β-cryptoxanthin is used in combination with vitamin C and/or vitamin E. Further, β-cryptoxanthin may be used for the purposes of this invention in combination with other carotenoids (e.g., β-carotene, lutein, zeaxanthin, lycopene, astaxanthin, canthaxanthin and/or apocarotenals), genistein, resveratrol, and (−)-epigallocatechin gallate (EGCG).

In accordance with the present invention, β-cryptoxanthin is suitably administered in dosages up to about 50 mg/day, more particularly, from about 100 μg/day to about 30 mg/day, especially from about 1 mg/day to about 10 mg/day for a human adult of about 70 kg body weight. If added as a supplement to animal feed, β-cryptoxanthin may be used in an amount to provide from about 100 to about 1000 ppm of β-cryptoxanthin in the final feed composition. If choleretic stimulants are co-administered, the amount of these may be in case of teas from about 1 to 5 cups per day, or in case of extracts, about 10 mg to about 500 mg per day for a human adult. If vitamin E is co-administered, the dosage of these is suitably from about from about 15 to about 500 mg vitamin E per day for a human adult. If vitamin C is co-administered, the dosage of that is suitably from about from about 50 to about 500 mg per day for a human adult.

For the purposes of the invention, β-cryptoxanthin is suitably provided in compositions for enteral application which may be solid or liquid galenical formulations, dietary compositions, animal feed or feed premixes for animals. Examples of solid galenical formulations are tablets, capsules (e.g. hard or soft shell gelatin capsules), pills, sachets, powders, granules and the like which contain the active ingredient together with conventional galenical carriers. Any conventional carrier material can be used. The carrier material can be organic or inorganic inert carrier material suitable for oral administration. Suitable carriers include water, gelatin, gum arabic, lactose, starch, magnesium stearate, talc, vegetable oils, and the like. Additionally additives such as flavouring agents, preservatives, stabilizers, emulsifying agents, buffers and the like may be added in accordance with accepted practices of pharmaceutical compounding. Additional active ingredients for co-administration with β-cryptoxanthin may administered, together with β-cryptoxanthin in a single composition, or may be administered in individual dosage units. Dietary compositions comprising β-cryptoxanthin can be beverages, instant beverages, or food/feed supplements.

As stated earlier, β-cryptoxanthin may be used in accordance with the present invention together with vitamin C or/and vitamin E, as well as in combination with other carotenoids (e.g., β-carotene, lutein, zeaxanthin, lycopene, astaxanthin, canthaxanthin, apocarotenals), genistein, resveratrol, and/or EGCG, which may be administered simultaneously with β-cryptoxanthin or separately. Furthermore, β-cryptoxanthin can be used in accordance with the present invention in combination with other vitamins and/or minerals conventionally used as food supplement, such as vitamins of the B group, and minerals containing Ca, Fe, P, Mg, Zn etc. A typical solid galenical formulation contains per dosage unit from about 1 μg to about 50 mg β-cryptoxanthin and, optionally, from about 15 mg (22 IU) to about 500 mg (750 IU) vitamin E. A typical liquid galenical formulation may contain, per ml, from about 10 ng to about 50 μg β-cryptoxanthin and, optionally, from about 10 mg (15 IU) to about 50 mg (75 IU) vitamin E. A typical dietary composition may contain from about 0.1 μg to about 5 mg β-cryptoxanthin and, optionally from about 1.5 mg (2.25 IU) to about 30 mg (45 IU) vitamin E per g of the total composition.

The efficacy of β-cryptoxanthin in promoting increased protein formation is evident from findings that upon administration of β-cryptoxanthin, genes involved in protein synthesis in skin, liver, kidney and spleen were up-regulated. In a six weeks study, β-cryptoxanthin beadlets were mixed into the diet of SKH-1 mice to provide 1200 ppm β-cryptoxanthin in the feed. Further, 0.125 g of sodium cholate per 100 g of feed was added to facilitate the uptake of β-cryptoxanthin. The average β-cryptoxanthin intake was 6.5 mg pro mouse and pro day. After six weeks, the effect of this dose on the overall gene expression was studied. β-cryptoxanthin-mediated gene expression modifications were assessed by GeneChip® DNA microarray technology. Total RNA was extracted from kidney, liver, skin and spleen using the TRIzol® method according to the manufacturer protocol. Reverse Transcription was performed with 10 μg total RNA followed by second strand synthesis of cDNA. The cDNA was then transcribed into multiple cRNA copies and labelled by biotin-coupled nucleotides and fragmented. 10 μg of labelled and fragmented cRNA were hybridized on Affymetrix Mice Genome 430A microarrays as described in the GeneChip® Expression Analysis Technical Manual (Affymetrix, Oxford, UK). For each β-cryptoxanthin-treated tissue, five replicates were performed and compared to six replicates of untreated control mice. After washing and staining procedures, the signals were amplified with a biotinylated anti-streptavidin antibody using the GeneChip® Fluidics Station. Subsequently, the microarrays were subjected to laser scanning and the hybridization signals were analyzed with Genedata Expressionist® and Phylosopher® software packages (Genedata, Basel, Switzerland). The primary goal was to identify genes with significant differences in expression between the control and the treated groups. Comparisons were made by computing the expression fold difference for each gene and listing those that showed greater than 1.20-fold increases or decreases in activity. Statistical analysis of GeneChip® arrays was performed using the Welch-test to identify compound-responsive genes in skin, kidney, liver and spleen. Statistical significance for the DNA microarrays was accepted at p-values<0.01.Genedata Phylosopher® software was used to assign the Affymetrix probe sequences to pathways and functional categories.

The results of this study focusing on the upregulation of genes which are involved in protein synthesis pathways are shown in Table 1.

Up-regulation of ribosomal proteins gene expression can be correlated with an increase of the overall protein synthesis insofar as ribosomes represent a highly efficient molecular machine catalizing protein synthesis (Al-Karadaghi et al., Prog. Biophys. Mol Biol, 2000, 73(2-4):167-93). Ribosomes are composed of two subunits, the large and the small one. Whereas the small subunit includes in bacteria 21 ribosomal proteins (S1-S21), the large subunit consists of 36 ribosomal proteins (L1-L36) (Wittmann H G., Annu Rev Biochem, 1982, 51:155-83). More recent data indicated that protein synthesis is accomplished by ribosomes acting in concert with tRNA and accessory factors to “translate” the genetic information contained in mRNA (Preiss et al., Bioessays, 2003, 25(12):1200-11). mRNA translation into protein can be divided into three successive phases: initiation, elongation and termination. Briefly, the initiation phase consists in the assembly of a ribosome with the initiator Met-tRNA at the start codon of the mRNA. The protein synthesis really begins during the elongation phase. When the ribosome reaches the stop codon, this terminates the protein synthesis by releasing the polypeptide, and probably the ribosome from the mRNA (Preiss et al., supra).

Thus the ribosome can be considered as a crucial component allowing protein synthesis to occur.

β-Cyptoxanthin enhances protein formation in muscle cell cultures

C2C12 cells from American Type Culture Collection (ATCC, CRL-1772).

The mouse myoblast cell line C2C12 was seeded into 6 cm² cell culture dishes at a density 2×10⁵ cells/per well. Cells were cultivated in the presence of Dulbecco's Minimum Essential Medium (DMEM) containing a final concentration of 10% fetal calf serum, 4500 mg/L glucose, 100 IU/ml Penicillin & 100 μg/ml Streptomycin 2 mM L-Glutamine and 1 mM sodium pyruvate. One day post seeding, cells were supplemented with fresh medium containing β-cryptoxanthin with a final concentration of 0.25 μM or 1 μM or vehicle (tetrahydrofurane). β-cryptoxanthin stock solution was prepared in tetrahydrofurane and the solvent concentration in the media was kept constant at 0.14% for all treatment conditions. Cultivation in medium containing β-cryptoxanthin followed up for a total of 7 consecutive days. The medium was changed every second day. On each day, cells from one well per treatment group were scraped into 0.4 ml of a lysis buffer consisting of 20 mM Tris pH 7.5, NaCl 150 mM, EDTA 1 mM, Nonidet 1%, proteinase inhibitors (Roche Molecular Systems, Mannheim Germany) and sonicated.

Aliquots of 10 μl of this solution were used for total protein, and 2 μl for total DNA analysis. After centrifugation for 5 min at 10000 g, supernatants were used for protein and DNA analysis in the soluble fraction.

Protein was determined using the BCA Protein Assay Reagent (Pierce, Rockford, USA). The Pico-green Assay (Molecular Probes, Leiden, The Netherlands) was used for analysis of DNA content.

The amount of protein was calculated as μg/well or in relation to the amount of cells present in the well, as indirectly determined by the amount of DNA/well. The results are shown in Tables 2 to 4.

TABLE 2
total protein μg/well after 1-7 days [d] in culture

| | [d] | | | | | | |
|---|---|---|---|---|---|---|---|
| | 1 | 2 | 3 | 4 | 5 | 6 | 7 |
| Vehicle | 707.2 | 674.8 | 707.2 | 903.6 | 1086 | 1082.4 | 950 |
| control | | | | | | | |
| 0.25 μM | 703.6 | 753.6 | 771.2 | 1136 | 1314.4 | 1411.2 | 1257.2 |
| β- | | | | | | | |
| crypto- | | | | | | | |
| xanthin | | | | | | | |
| 1 μM | 750 | 757.2 | 750 | — | 1236 | 1232.4 | 1193.2 |
| β- | | | | | | | |
| crypto- | | | | | | | |
| xanthin | | | | | | | |

TABLE 3
soluble protein μg/well after 1-7 days [d] in culture

| | [d] | | | | | | |
|---|---|---|---|---|---|---|---|
| | 1 | 2 | 3 | 4 | 5 | 6 | 7 |
| Vehicle | 671 | 540 | 619 | 760 | 940 | 943 | 860 |
| control | | | | | | | |
| 0.25 μM | 536 | 592 | 626 | 915 | 1091 | 1267 | 1284 |
| β- | | | | | | | |
| cryptoxanthin | | | | | | | |
| 1 μM | 574 | 547 | 609 | 857 | 1126 | 1146 | 1118 |
| β- | | | | | | | |
| cryptoxanthin | | | | | | | |

TABLE 4
μg soluble protein/μg DNA after 1-7 days [d] in culture

| | [d] | | | | | | |
|---|---|---|---|---|---|---|---|
| | 1 | 2 | 3 | 4 | 5 | 6 | 7 |
| Vehicle | 299 | 228 | 256 | 339 | 429 | 471 | 483 |
| control | | | | | | | |
| 0.25 μM | 227 | 244 | 274 | 406 | 505 | 713 | 693 |
| β- | | | | | | | |
| cryptoxanthin | | | | | | | |
| 1 μM | 246 | 231 | 265 | 382 | 539 | 624 | 644 |
| β- | | | | | | | |
| cryptoxanthin | | | | | | | |

The data from the above Tables demonstrate that β-cryptoxanthin showed an approx. 1.4 fold increase in total protein (Table 2), an increase in soluble protein (Table 3) and an increase in the specific amount of protein/μg DNA (Table 4) at the concentration of 0.25 μM. The effect plateaued at 1 μM.

Accordingly, the above data show that β-cryptoxanthin is an effective enhancer of protein formation in muscle cells.

The invention is illustrated further by the Examples which follow.

### EXAMPLE 1

Beadlets comprising the ingredients as indicated (wt.-%) can be prepared using conventional technology:

| | |
|---|---|
| β-Cryptoxanthin | 5.0 |
| Gelatine 140 Bloom | 32.5 |
| Sucrose | 31.5 |
| Na-ascorbat | 2.0 |
| Ascorbyl palmitate | 2.0 |
| dl-alpha tocopherol | 1.0 |
| Fluid corn starch | 25 |

The beadlets are directly mixed into animal feed at concentrations up to 1200 mg of β-cryptoxanthin/kg of feed.

### EXAMPLE 2

A tablet is formulated to contain:

Active ingredients:

| | | |
|---|---|---|
| β-cryptoxanthin | 15 | mg |
| dl-alpha tocopherol | 300 | mg |

Excipients:

| | | |
|---|---|---|
| lactose powder | | |
| saccharose | | |
| magnesium stearate | | |
| gelatin | ad 500 | mg |

For the treatment of chronic diseases (chronic renal failure, chronic heart insufficiency, chronic obstructive pulmonary disease, tumor cachexia, sarcopenia) one tablet per day may be administered to a human adult.

### EXAMPLE 3

A capsule is prepared containing the following ingredients:

Active ingredients:

| | | |
|---|---|---|
| β-cryptoxanthin | 5 | mg |
| dl-alpha tocopherol | 100 | mg |

Excipients:

| | | |
|---|---|---|
| gelatin | | |
| lactose | | |
| magnesium stearate | | |
| rice starch | | |
| glycerol palmitostearate | ad 300 | mg |

For the treatment of chronic diseases (chronic renal failure, chronic heart insufficiency, chronic obstructive pulmonary disease, tumor cachexia, sarcopenia) one to three capsules per day may be administered to a human adult.

### EXAMPLE 4

An Infant Formula prepared with the following components may contain per 100 g:

| | | |
|---|---|---|
| β-cryptoxanthin | 1 | mg |
| Total fat | 3.7 | g |
| Sodium | 19 | mg |
| Potassium | 76 | mg |
| Total carbohydrate | 7.7 | g |
| Protein | 1.5 | g |

Vitamins and minerals:

| | | |
|---|---|---|
| vitamin A | 210 | IU |
| vitamin C | 8.4 | mg |
| vitamin D | 42 | IU |
| vitamin E | 1.5 | IU |
| vitamin K | 5.6 | μg |
| thiamin (B1) | 56.3 | μg |
| riboflavin (B2) | 63.4 | μg |
| niacin (B3) | 704.2 | μg |
| vitamin B6 | 42.2 | μg |
| folate, folic acid, folacin | 11.3 | μg |
| vitamin B12 | 0.2 | μg |
| biotin | 2.1 | μg |
| pantothenic acid | 352.1 | mg |
| calcium | 54.9 | mg |
| iron | 1.3 | mg |
| phosphorus | 37.3 | mg |
| iodine | 7 | μg |
| magnesium | 5.6 | mg |
| zinc | 0.7 | mg |
| selenium | 2 | μg |
| copper | 52.8 | μg |
| manganese | 10.6 | μg |
| chloride | 44.4 | mg |
| potassium | 76 | mg |
| choline | 8.4 | mg |
| inositol | 4.2 | mg |
| linoleic acid | 605.6 | mg |
| water | 94.4 | g |

After mixing of all the mentioned components, the infant formula was lyophilized to become a powder. Infants from 1 to 12 months may receive 10 g of infant formula per kg body weight per day.

### EXAMPLE 5

An energy drink of 250 ml may contain:

| | | |
|---|---|---|
| β-cryptoxanthin | 5 | mg |
| proteins | 3.9 | g |
| lipids | 14.3 | g |
| carbohydrates | 71 | g |
| H₂O ad 250 ml | | |

Vitamin and minerals:

| | | |
|---|---|---|
| vitamin E | 5 | mg |
| vitamin B1 | 1.2 | mg |
| vitamin B2 | 1.2 | mg |
| vitamin B6 | 1.2 | mg |
| vitamin C | 45 | mg |
| niacin | 9 | μg |
| sodium | 190 | mg |
| potassium | 180 | mg |
| calcium | 70 | mg |
| phosphor | 120 | mg |
| magnesium | 30 | mg |
| iron | 1.3 | mg |
| zinc | 1.6 | mg |

Teenagers and young people typically consume 100-500 ml of energy drink per day.

### EXAMPLE 6

An energy bar of 25 g may contain:

| | | |
|---|---|---|
| β-cryptoxanthin | 2.5 | mg |
| proteins | 1 | g |
| lipids | 3.5 | g |
| carbohydrates | 16.5 | g |
| fibres | 3 | g |

Vitamins and minerals:

| | | |
|---|---|---|
| vitamin E | 5 | mg |
| vitamin B1 | 1.2 | mg |
| vitamin B2 | 1.2 | mg |
| vitamin B6 | 1.2 | mg |
| vitamin C | 45 | mg |
| niacin | 11 | mg |
| sodium | 190 | mg |
| potassium | 180 | mg |
| chloride | 210 | mg |
| calcium | 70 | mg |
| phosphor | 120 | mg |
| magnesium | 30 | mg |
| iron | 1.3 | mg |
| zinc | 1.6 | mg |

Teenagers and young people typically consume 1-3 energy bars per day.

### EXAMPLE 7

A sport drink of 250 ml contains:

| | | |
|---|---|---|
| β-cryptoxanthin | 10 | mg |
| proteins | 3.9 | g |
| lipids | 14.3 | g |
| carbohydrates | 71 | g |
| H₂O ad 250 ml | | |

Vitamins and minerals:

| | | |
|---|---|---|
| vitamin E | 5 | mg |
| vitamin B1 | 1.2 | mg |
| vitamin B2 | 1.2 | mg |
| vitamin B6 | 1.2 | mg |
| vitamin C | 45 | mg |
| niacin | 9 | mg |
| sodium | 190 | mg |
| potassium | 180 | mg |
| calcium | 70 | mg |
| phosphor | 120 | mg |
| magnesium | 30 | mg |
| iron | 1.3 | mg |
| zinc | 1.6 | mg |

An athlete or bodybuilder weighing 90 kg typically consumes 250 to 500 ml of sports drink daily. This drink is also suitable for elderly people to prevent or ameliorate sarcopenia.

### EXAMPLE 8

A feed premix for poultry contains per kg

| | | |
|---|---|---|
| Vitamin A (Rovimix ® A 500) | 2 | g |
| Vitamin D3 (Rovimix ® D3 500) | 1 | g |
| Vitamin E (Rovimix ® E 50 Ads) | 4 | g |
| Vitamin B12 (B12 1%) | 0.1 | g |
| Vitamin B3 (Rovimix ® B2 80-SD) | 0.5 | g |
| Niacin (Rovimix ® Niacin) | 0.1 | g |
| Calpan (Rovimix ® Calpan) | 0.05 | g |
| Folic acid (Rovimix ® Folic 80 SD) | 2 | mg |
| Vitamin B6 (Rovimix ® B6) | 8 | mg |
| Vitamin B1 (Rovimix ® B1) | 4 | mg |
| Vitamin C | 0.2 | g |
| Carotenoids (Carophyll ® Red) | 2 | g |
| β-cryptoxanthin beadlet formulation 5% | 2 | g |
| (see Example 1) | | |
| Choline chloride 50% | 150 | g |
| Mn(IV)oxide | 30 | g |
| Zn oxide | 6 | g |
| Fe(II)sulfate monohydrate | 10 | g |
| Cu(II)oxide | 1 | g |
| Co(II)sulfate | 0.1 | g |
| Carrier | 792 | g |
| (Limestone, rice hulls, wheat middlings) | | |
| Total | 1000 | g |

Animal feed is supplemented with 1 to 30 g feed premix per kg feed.

1. The use of β-cryptoxanthin in the manufacture of a composition for promoting an increased protein formation and/or prevention of loss of protein in human or an animal.

2. The use as in claim 1 wherein the composition is for promoting increased protein formation and/or prevention of loss of proteins due to an adverse health condition.

3. The use as in claim 2 wherein the adverse health condition is a chronic disease, tumor cachexia, or an eating disorder.

4. The use as in claim 2 wherein the adverse health condition is a viral infection.

5. The use as in claim 2 wherein the adverse health condition is a status after second or third degree burn, after myocardial infarction, or after organ transplant.

6. The use as in claim 2 wherein the adverse health condition is a status of immobilization after skeletal injury or surgery.

7. The use as in claim 1 wherein the composition is for fostering growth and development.

8. The use as in claim 1 wherein the composition is for promoting an increased protein formation in sports and workout activities.

9. The use as in claim 1 wherein the composition is for promoting an increased protein formation and/or prevention and treatment of sarcopenia during aging.

10. The use as in claim 1 wherein the composition is for promoting growth in farm animals and pets, and for enhancing performance of competitive sports animals.

11. The use as in claim 1 wherein the composition is a galenical composition.

12. The use as in claim 1 wherein the composition is a dietary composition.

13. The use as in claim 12 wherein the dietary composition is a feed or feed premix.

14. The use as in claim 1 wherein the β-cryptoxanthin is all-E β-cryptoxanthin.

15. The use as in claim 11 wherein the amount of β-cryptoxanthin in the composition is up to about 50 mg per dosage unit of a solid composition and up to about 50 μg per ml of a liquid composition.

16. The use as in claim 12 wherein the amount of β-cryptoxanthin in the composition is up to about 5 mg per g of the total composition.

17. The use as in claim 1 wherein the composition additionally contains at least one of vitamin E and vitamin C.

18. A method for promoting an increased protein formation and/or prevention of loss of protein in human or an animal which comprises administering to said human or animal an effective amount of β-cryptoxanthin.

19. The method of claim 18 to improve an adverse health condition.

20. The method as in claim 19 wherein the adverse health condition is a chronic disease, tumor cachexia, or an eating disorder.

21. The method as in claim 19 wherein the adverse health condition is a viral infection.

22. The method as in claim 19 wherein the adverse health condition is a status after second or third degree burn, after myocardial infarction, or after organ transplant.

23. The use as in claim 19 wherein the adverse health condition is a status of immobilization after skeletal injury or surgery.

24. The method as in claim 18 wherein the increased protein formation is to foster growth and development.

25. The method as in claim 18 for promoting an increased protein formation in sports and workout activities.

26. The method as in claim 18 for promoting an increased protein formation and/or prevention of loss of protein during aging.

27. The method as in claim 18 wherein the increased protein formation is to promote growth of farm animals and pets, and to enhance performance of competitive sports animals.

28. The method as in claim 18 wherein up to about 50 mg of β-cryptoxanthin, more particularly, from about 100 μg/day to about 30 mg/day, especially from about 1 mg/day to about 10 mg/day are administered to a human adult of about 70 kg body weight.

29. The method as in claim 18 wherein a bile salt or choleretic stimulant is co-adminstered.
